(19) United States
(12) Patent Application Publication
(10) Pub. No.: US 2011/0028852 A1
(43) Pub. Date: Feb. 3, 2011

(54) Implantable Pressure Sensor with Membrane Bridge

(76) Inventor: Arshad A. Alfoqaha, Eden Prairie, MN (US)

Correspondence Address: Medtronic, Inc., 710 Medtronic Parkway, Mail Stop LC340, Minneapolis, MN, 55432, US

(21) Appl. No.: 12/512,869

(22) Filed: Jul. 30, 2009

(51) Int. Cl. A61B 5/02; B23K 26/00

(52) U.S. Cl. 600/486; 219/121.63

(57) ABSTRACT

An implantable pressure sensor having improved bend error performance is provided having a capsule housing a pressure sensing device, wherein the capsule includes an opening that allows the pressure sensing device to obtain pressure measurements from an environment surrounding the capsule. A rigid bridge is attached to the capsule so as to extend across the opening of the capsule. The bridge includes at least one opening that exposes the pressure sensing device to the surrounding environment. The rigidity of the bridge functions to prevent deformations from bending forces that are exerted on the sensor and also functions to divert loads from such bending forces away from the pressure sensing device to instead travel through the bridge, which in turn resists deformation. A fill material may be situated between the bridge and capsule so as to prevent bodily fluid or tissue from building up in the sensor under the bridge.

### TECHNICAL FIELD

This disclosure relates to an implantable pressure sensor for providing pressure measurements and more particularly to an implantable pressure sensor with a membrane bridge for reducing bend error in the pressure measurements.

### BACKGROUND

Historically, an implantable, subcutaneous or external medical device may be used to monitor physiological parameters in a patient to ensure that they fall within certain acceptable values or ranges. Such medical devices have further been capable of delivering therapy to a patient, where the device may be configured to automatically deliver the therapy in response to the monitored physiological parameters reaching certain values or ranges. Implantable pressure transducers and sensors have been developed for temporary or chronic use in a body organ or vessel for measuring pressure or taking other readings in implanted locations within a patient's body. Such implantable pressure sensors are sometimes attached to leads inserted within the patient's body or connected to implantable medical devices for providing pressure measurements in implanted or positioned locations for use in monitoring pressure-related physiological parameters, diagnosing conditions or determining therapy that may be required.

### SUMMARY

In one or more embodiments, an implantable pressure sensor having improved bend error performance is provided that includes a capsule having an opening that allows a pressure sensing device contained within the capsule to obtain pressure measurements from an environment surrounding the capsule. A rigid bridge member is attached to the capsule so as to extend across the opening of the capsule. The bridge member includes at least one opening that exposes the pressure sensing device to the surrounding environment. The rigidity of the bridge member functions to reduce or prevent deformations from bending forces or strain that are exerted on the implantable pressure sensor and also functions to divert loads from such bending forces or strain away from the pressure sensing device contained within the capsule to instead travel through the bridge member, which in turn resists deformation. In this manner, the bridge member functions to reduce bend error in the pressure measurements obtained by the pressure sensing device by preventing or substantially reducing deformation in the pressure sensing device that can result from the bending loads and strain exerted on the implantable pressure sensor.

In one or more embodiments, the pressure sensing device includes a membrane positioned adjacent to the opening of the capsule so that pressures from the surrounding environment will act upon the membrane through the opening. The membrane is a component of at least one capacitor structure used by the pressure sensing device in generating capacitive measurements indicative of the pressure of the surrounding environment. Pressures exerted on the membrane will in turn cause a corresponding movement of membrane which in turn will alter the measured capacitance. A fill material, such as silicon or the like, may be situated above the membrane in the opening in the bridge member and/or in any gaps between the bridge member and the capsule so as to prevent bodily fluid or tissue from building up in implantable pressure sensor between the bridge member and the membrane.

Many other features and embodiments of the present invention will be apparent from the accompanying drawings and from the following detailed description.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1 illustrates a representative example of an implantable pressure sensor connected to a lead positioned in a heart in accordance with one or more embodiments of the present disclosure.

FIG. 2 illustrates a partially exploded perspective view of an implantable pressure sensor in accordance with one or more embodiments of the present disclosure.

FIG. 3 illustrates a perspective view of an implantable pressure sensor in accordance with one or more embodiments of the present disclosure with the membrane bridge being attached.

FIG. 4 illustrates a perspective view of an implantable pressure sensor in accordance with one or more embodiments of the present disclosure having the injected fill material.

FIG. 5 illustrates a side, partial cross-sectional view of the implantable pressure sensor of FIG. 3 taken generally along lines V-V.

FIG. 6 illustrates a top view of the implantable pressure sensor of FIG. 4.

FIG. 7 illustrates an end, partial cross-sectional view of the implantable pressure sensor of FIG. 4 taken generally along lines VII-VII.

### DETAILED DESCRIPTION

In the following detailed description of embodiments of the present disclosure, reference is made to the accompanying drawings in which like elements in the figures are provided like reference numerals, and in which is shown by way of illustration specific embodiments in which the present disclosure may be practiced. These embodiments are described in sufficient detail to enable those skilled in the art to practice the present disclosure, and it is to be understood that other embodiments may be utilized and that logical, mechanical, electrical, functional, and other changes may be made without departing from the scope of the present disclosure. The following detailed description is, therefore, not to be taken in a limiting sense, and the scope of the present disclosure is defined only by the appended claims. As used in the present disclosure, the term “or” shall be understood to be defined as a logical disjunction and shall not indicate an exclusive disjunction unless expressly indicated as such or notated as “xor.”

Furthermore, reference in this specification to “one embodiment”, “an embodiment”, “other embodiments”, or the like means that a particular feature, structure, or characteristic described in connection with the embodiment is included in at least one embodiment of the present disclosure. The appearances of, for example, the phrase “in one embodiment” in various places in the specification are not necessarily all referring to the same embodiment, nor are separate or alternative embodiments mutually exclusive of other embodiments. Moreover, whether or not there is express reference to an “embodiment” or the like, various features are described which may be exhibited by some embodiments and not by others. Similarly, various requirements are described which may be requirements for some embodiments but not other embodiments.

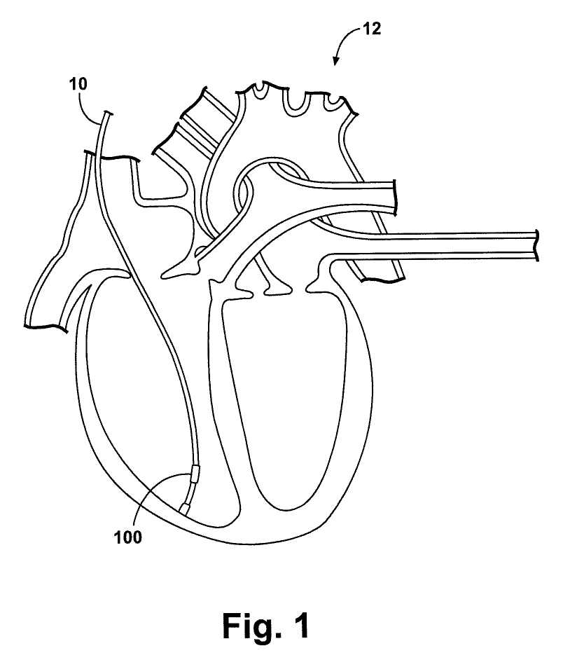

An implantable pressure sensor having improved bend error performance is provided. In one or more embodiments, the implantable pressure sensor may be attached to a lead or may be a component of a lead that is implanted or inserted into the body of a person, such as a transvenous, ventricular lead having an absolute pressure sensing capability that provide signals indicative of pressure to an Implantable Medical Device (IMD) attached to the lead. Examples of such transvenous, ventricular leads include Chronicle® Pressure Sensing Leads (PSLs), Model 4328A or 4328B Pressure Sensing Leads, available from Medtronic, Inc., Minneapolis, Minn. In one or more embodiments, the implantable pressure sensor may comprise an endocardial lead for implantation in a body organ (e.g., heart chamber) or cardiac blood vessel for sensing blood pressure and providing blood pressure signals to an implanted or external hemodynamic monitor and/or therapy delivery device. For example, FIG. 1 illustrates a representative example of an implantable pressure sensor 100 connected to a lead 10 positioned in a heart 12. In other embodiments, the implantable pressure sensor may otherwise be connected to IMDs for providing pressure measurements in implanted or positioned locations for use in monitoring pressure-related physiological parameters, diagnosing conditions or determining therapy that may be required.

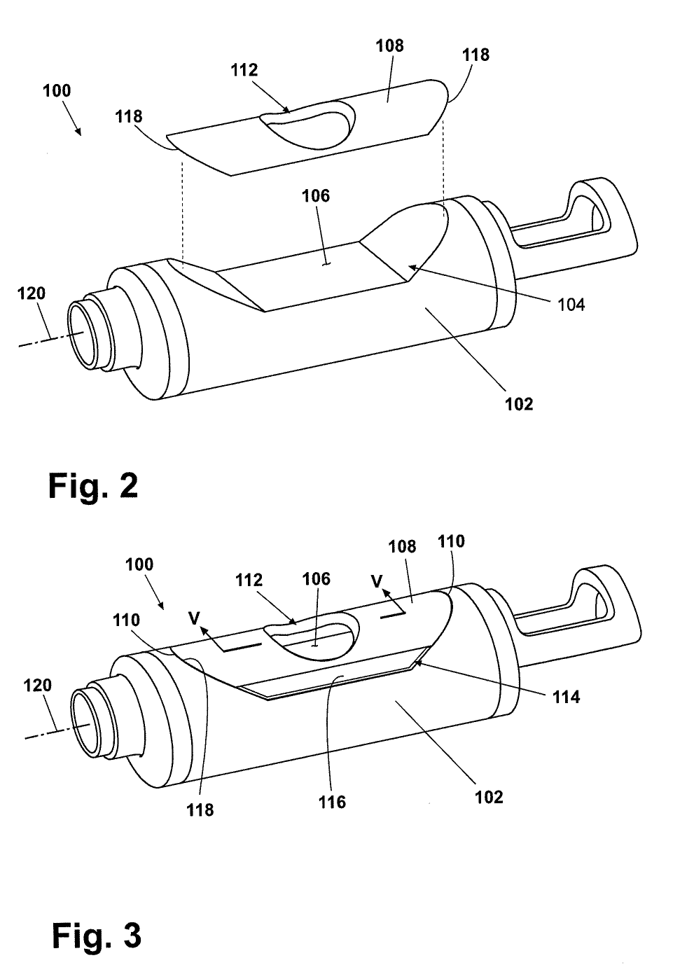

FIG. 2 illustrates an exploded perspective view of an implantable pressure sensor 100 formed in accordance with one or more embodiments of the present disclosure. Sensor 100 includes a capsule 102 having an opening 104 that allows a pressure sensing device 106 (not shown in its entirety) contained within capsule 102 to obtain pressure measurements in an environment surrounding capsule 102. By way of example only and without limitation, capsule 102 and pressure sensing device 106 may comprise a MEMS-based Pascal pressure sensing capsule available from Ansys Inc. of Canonsburg, Pa. or may comprise implantable pressure sensors described in commonly assigned U.S. Pat. No. 6,221,024, entitled “Implantable Pressure Sensor and Method of Fabrication” and U.S. Pat. No. 6,234,973, entitled “Implantable Medical Device for Sensing Absolute Blood Pressure and Barometric Pressure,” the contents of each and all of which are hereby incorporated by reference in their entireties.

In one or more embodiments, capsule 102 can be formed of material comprising titanium, stainless steel, MP35N alloy (a nonmagnetic, nickel-cobalt-chromium-molybdenum alloy), platinum, other bio-compatible metals, silicone rubber, polyurethane, epoxy, acetyl co-polymer plastics, other bio-compatible plastics (e.g., PolyEtherEtherKetone (PEEK), liquid crystal polymer (LCP) plastics, etc.) or any combination of the aforementioned materials.

In one or more embodiments, a rigid bridge member 108 (hereafter referred to as bridge 108) is attached to capsule 102 so as to extend across opening 104 of capsule 102, as illustrated in the attached configuration of FIG. 3. In one or more embodiments, bridge 108 comprises titanium (Ti), stainless steel, MP35N alloy (a nonmagnetic, nickel-cobalt-chromium-molybdenum alloy), platinum, another bio-compatible metal or any combination thereof, where bridge 108 is welded or otherwise rigidly adhered to capsule 102 at attachment points 110. Bridge 108 includes at least one opening 112 that exposes the pressure sensing device 106 to the surrounding environment. In one or more embodiments, bridge 108 may also be shaped so that at least one gap 114 between bridge 108 and capsule 102 exists between the components when attached together to further expose pressure sensing device 106 to pressures from the environment surrounding sensor 100. Opening 112 and gap(s) 114 allow pressure sensing device 106 to obtain pressure measurements from the environment surrounding sensor 100 with bridge 108 being attached to capsule 102.

In one or more embodiments, pressure sensing device 106 includes a membrane 116 or diaphragm positioned adjacent to opening 104 of capsule 102 so that pressures from the surrounding environment will act upon the membrane through opening 104. Since membrane 116 is the only portion of pressure sensing device 106 that is exposed to the surrounding environment that can be seen in the perspective views of the drawings, membrane 116 will be hereafter referred to in reference to the drawings when describing both membrane 116 and pressure sensing device 106. It is understood that in accordance with one or more embodiments, membrane 116 is a component of at least one capacitor structure used in generating capacitive measurements indicative of the pressure of the surrounding environment. Pressures exerted on membrane 116 will in turn cause a corresponding movement of membrane 116 which in turn will alter a measured capacitance, where the measured capacitance corresponds to the pressure from the surrounding environment acting on membrane 116.

Implantable pressure sensors 100, for instance piezo-pyroluminescent (PPL) pressure sensors, that are attached to leads are generally subject to high bend errors in the pressure measurements resulting from bending strain and forces that are exerted on the sensor 100 that can deform membrane 116 and other components of pressure sensing device 106. Bend errors can cause inaccurate and faulty pressure measurements to be obtained by the sensor 100.

In order to provide improved bend error performance by sensor 100, the rigidity of the attached bridge 108 functions to reduce bending deformations from bending forces that are exerted on implantable pressure sensor 100. Bridge 108 further functions to divert loads from such bending forces away from membrane 116 and other portions of pressure sensing device 106 contained within capsule 102 to instead travel through the bridge 108, where bridge 108 is formed of a rigid material that resists deformation. In this manner, the bridge 108 functions to reduce bend error in the pressure measurements obtained by the pressure sensing device 106 by preventing or substantially reducing the bending loads and strain exerted on membrane 116 and other portions of pressure sensing device 106, thereby preventing or substantially reducing deformation of membrane 116 from such bending loads and strain.

In one or more embodiments, bridge 108 may be formed to matingly engage capsule 102 in opening 104 to further enhance the rigidity of the overall sensor 100 and also to assist in distributing forces and loads between bridge 108 and capsule 104. For example, the side surfaces 118 of bridge 108 at the attachment points 110 to capsule 102 may be formed to be slanted with a substantially curved perimeter, where opening 104 is formed to possess a corresponding shape for receiving bridge 108 and for distributing forces in a plurality of directions. However, the shape of capsule 102 and bridge 108 is not limited to this shape or the ones illustrated in the attached figures, where the shape of capsule 102 and bridge 108 may comprise any shape that is configured to optimally distribute bend forces and strain across the capsule 102 and bridge 108 and away from membrane 116.

In one or more embodiments, the attachment points 110 for attaching bridge 108 to capsule 102 are selected to substantially coincide along a longitudinal directional axis 120 of a lead 10 to which sensor 100 is attached, since the bending forces or strain exerted on sensor 100 can often result from bending of the lead 10 along its longitudinal directional axis 120.

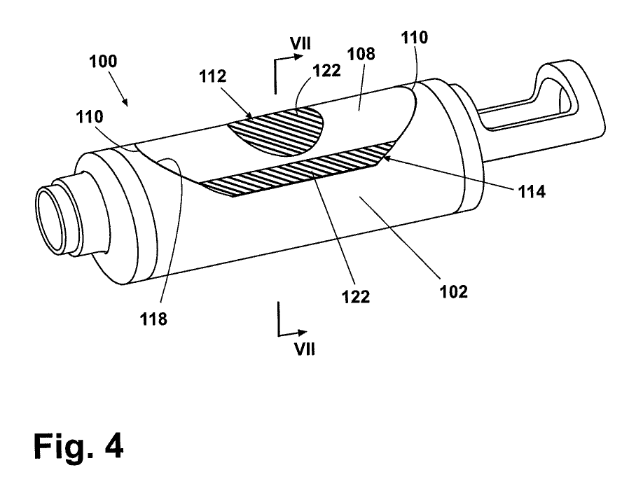

In one or more embodiments, with reference to FIG. 4, a fill material 122 may be used to fill the area 124 between membrane 116 and bridge 108 and also to fill opening 112 in bridge 108 and/or any gap(s) 114 between the bridge 108 and capsule 102 so as to prevent bodily fluid or tissue from the surrounding environment from entering into and building up within implantable pressure sensor 100 between bridge 108 and membrane 116. In one or more embodiments, a soft fill material 122 such as silicone or Nusil 1137 silicone (which is available from Nusil Silicone Technology Incorporated, Carpinteria, Calif.) may be injected into the fill area 124 using, for example, silicone injection molding technology (LSR). The fill material 122 may be selected to possess a certain thickness such that implantable pressure sensor 100 becomes impervious to hydration damage or any deformation effects caused by soaking or hydration from bodily fluids. In one or more embodiments, fill material 122 is formed to possess a thickness of approximately 5 mils, while it is understood that the selected thickness may vary depending upon the desired characteristics of the sensor 100. The use of bridge 108 coupled with the injection of a soft fill material 122 into any additional openings in and around bridge 108 further eliminates the risk of membrane 116 deformation, gap changes or hydration damage that could be caused by soaking, which in turn further significantly reduces bend error. As a result, the resulting implantable pressure sensor 100 is made much more stable and resolute.

In one or more embodiments, the size and/or shape of opening 112 in bridge 108 can be adjusted depending on the sensitivity, desired operational characteristics, and surrounding environment of the implantable pressure sensor 100. For example, if a higher degree of sensitivity to the surrounding environment is required, the size of opening 112 can be selected to be larger. Conversely, if only a lower degree of sensitivity is required, the size of opening 112 can be selected to be smaller. Along these same lines, in one or more embodiments, the shape of bridge 108 and capsule 102 can be selected to form gap(s) 114 of a desirable shape and size to provide desired sensitivity, operational characteristics, and resistivity to fluid/tissue build up from the surrounding environment.

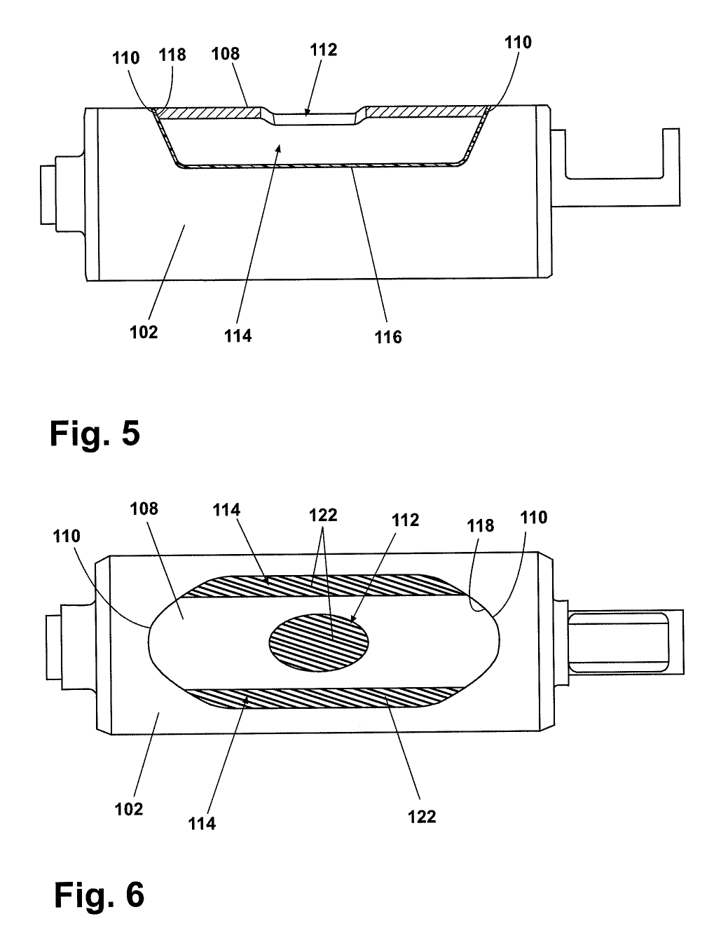

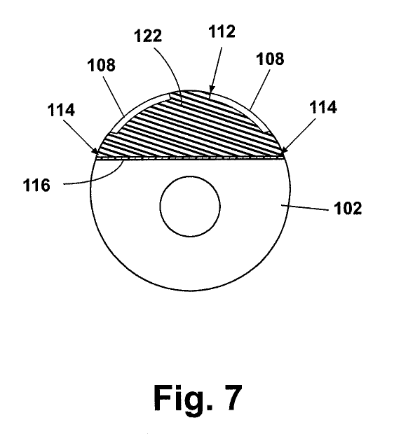

FIGS. 5-7 illustrate additional views of the implantable pressure sensor 100 illustrated in FIGS. 3 and 4, where like elements illustrated in FIGS. 5-7 of implantable pressure sensor 100 have been described above and a redundant description of such elements will not be repeated. FIG. 5 illustrates a side, partial cross-sectional view of the implantable pressure sensor 100 in accordance with one or more embodiments. Like elements illustrated in FIG. 4 of implantable pressure sensor 100 have been described above and a redundant description of such elements will not be repeated. In the side view of FIG. 4, the bridge 108 is more clearly seen as “bridging” two sides of capsule 102 (the two inner surfaces of the capsule 102 facing inwards towards the bridge 108) between connection points 110. FIG. 6 illustrates a top view of the implantable pressure sensor 100 of FIG. 4, where it can be seen that in one or more embodiments, gap(s) 114 between capsule 102 and bridge 108 may be formed to exist on two or more sides of bridge 108. FIG. 7 is an end, partial cross-sectional view of the implantable pressure sensor 100 in accordance with one or more embodiments

Overall advantages of the present disclosure include being able to position a rigid bridge 108 over a membrane 116 of a pressure sensing device 106 in an implantable pressure sensor 100 in order to reduce bend error problems and improve the overall accuracy and performance of the implantable pressure sensor 100. Furthermore, by filling the openings between capsule 102 and bridge 108 and over membrane 116 with a soft fill material 122, membrane deformations, gap changes, and hydration damage caused by soaking from bodily fluids can be eliminated as well as eliminating the build-up of tissue and bodily fluids between bridge 108 and capsule 102.

While an implantable pressure sensor with a membrane bridge has been described in terms of what are presently considered to be the most practical and preferred embodiments, it is to be understood that the present disclosure need not be limited to the above embodiments. It should also be understood that a variety of changes may be made without departing from the essence of the invention. Such changes are also implicitly included in the description and still fall within the scope of the present disclosure. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element of the invention may be expressed by equivalent apparatus terms or method terms. Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action. Similarly, each physical element disclosed should be understood to encompass a disclosure of the action which that physical element facilitates.

The above is intended to cover various modifications and similar arrangements included within the spirit and scope of the below appended claims, the scope of which should be accorded the broadest interpretation so as to encompass all such modifications and similar structures and/or method steps. Therefore, the present invention includes any and all embodiments of the following below appended claims.

1. An implantable pressure sensor, comprising:
a capsule housing a pressure sensing device, wherein the capsule includes an opening that allows the pressure sensing device to obtain pressure measurements from an environment surrounding the capsule; and
a rigid bridge member is attached to the capsule so as to extend across the opening of the capsule.

2. The implantable pressure sensor of claim 1, wherein the bridge member includes at least one opening that allows the pressure sensing device to obtain pressure measurements from an environment surrounding the capsule.

3. The implantable pressure sensor of claim 1, further comprising at least one gap positioned between the bridge member and the capsule that allows the pressure sensing device to obtain pressure measurements from an environment surrounding the capsule.

4. The implantable pressure sensor of claim 1, wherein the bridge member is formed of a material comprising at least one of titanium, stainless steel, MP35N alloy, platinum or another bio-compatible metal.

5. The implantable pressure sensor of claim 2, further comprising a soft fill material positioned at least in the opening in the bridge member.

6. The implantable pressure sensor of claim 5, wherein the soft fill material is further positioned to occupy an area above the pressure sensing device between the bridge member and the capsule.

7. The implantable pressure sensor of claim 5, wherein the soft fill material comprises a silicone material.

8. The implantable pressure sensor of claim 1, wherein the bridge member and opening in the capsule are configured so as to distribute bending forces exerted on the implantable pressure sensor through the bridge member and away from the pressure sensing device.

9. A membrane bridge for reducing bend error in an implantable pressure sensor, comprising:
a rigid bridge member attachable to a capsule housing a pressure sensing device including a membrane that is responsive to pressure measurements from an environment surrounding the capsule, wherein the capsule includes an opening adjacent to the membrane that allows the pressure sensing device to obtain pressure measurements from the environment surrounding the capsule,
wherein the rigid bridge member is formed to extend across the opening of the capsule over the membrane.

10. The membrane bridge of claim 9, wherein the bridge member includes at least one opening that exposes the membrane of the pressure sensing device to the environment surrounding the capsule for obtaining pressure measurements.

11. The membrane bridge of claim 10, wherein the bridge member is shaped so as to further form at least one gap positioned between the bridge member and the capsule that further exposes the membrane of the pressure sensing device to the environment surrounding the capsule for obtaining pressure measurements.

12. The membrane bridge of claim 9, wherein the bridge member is formed of a material comprising at least one of titanium, stainless steel, MP35N alloy, platinum or another bio-compatible metal.

13. The membrane bridge of claim 9, wherein the bridge member is shaped to matingly engage the opening in the capsule so as to distribute bending forces exerted on the implantable pressure sensor through the bridge member and away from the pressure sensing device.

14. A method for forming an implantable pressure sensor having reduced bend errors in measured pressures, the method comprising:
providing an implantable pressure sensor having a capsule housing a pressure sensing device that is responsive to pressure measurements from an environment surrounding the capsule, wherein the capsule includes an opening adjacent to the pressure sensing device for exposing the pressure sensing device to pressures from the environment surrounding the capsule,
attaching a rigid bridge member to the capsule to extend across the opening of the capsule so as to distribute bending forces exerted on the implantable pressure sensor through the bridge member and away from the pressure sensing device.

15. The method of claim 14, further comprising forming the bridge member to possess at least one opening that allows the pressure sensing device to obtain pressure measurements from an environment surrounding the capsule.

16. The method of claim 14, further comprising configuring the bridge member such that when attached to the capsule at least one gap exists between the bridge member and the capsule that allows the pressure sensing device to obtain pressure measurements from an environment surrounding the capsule.

17. The method of claim 14, further comprising forming the bridge member of a material comprising at least one of titanium, stainless steel, MP35N alloy, platinum or another bio-compatible metal.

18. The method of claim 15, further comprising filling at least the opening in the bridge member with a soft fill material.

19. The method of claim 16, further comprising filling the at least one gap between in the bridge member and the capsule with a soft fill material.

20. The method of claim 14, further attaching the bridge member to the capsule by laser welding the components together at selected locations around the opening of the capsule.
